(19) United States
(12) Patent Application Publication
(10) Pub. No.: US 2024/0023503 A1
(43) Pub. Date: Jan. 25, 2024

(54) APPARATUS AND METHOD FOR ALGAE GROWTH

(71) Applicant: Arthur DEANE, Calgary (CA)

(72) Inventor: Arthur DEANE, Calgary (CA)

(21) Appl. No.: 18/257,112

(22) PCT Filed: Jan. 6, 2022
(86) PCT No.: PCT/CA2022/050008

§ 371 Date: Jun. 13, 2023

Related U.S. Application Data
(60) Provisional application No. 63/134,439, filed on Jan. 6, 2021.

(51) Int. Cl. A01G 33/00; C12M 1/00; C12M 1/12; C12M 1/06

(52) U.S. Cl. CPC A01G 33/00; C12M 23/38; C12M 23/06; C12M 21/02; C12M 27/02

(57) ABSTRACT

An apparatus for growing algae includes a tank for containing growth media, a transparent lid for closing an open top of the tank, and an impeller coupled to a barrel within the tank. A method for growing algae includes swirling a growth media in a tank such that the growth media circulates around the tank passing by a dark section of the tank and an illuminated section of the tank.

### TECHNICAL FIELD

The present invention relates to commercial farming apparatus and methods in general, and apparatus and methods for algae growth in particular.

### BACKGROUND

Commercial farming, including the growing and harvesting of algae, is of interest because the produced algae can have a variety of uses. For example, algae can produce bio-mass feed stocks for the production of bio-fuels such as: bio-diesel, bio-kerosene, bio-ethanol, human food, animal feed, wastewater treatment, and the like.

One common way of commercially growing algae is to use an open system where the algae growth process is open to the elements. These open pond systems can best be described as a number of plastic or clay lined shallow dugouts, occupying hundreds of hectares of land area. Race track systems are one type of known open pond system.

The open pond algae grow operations do not allow control over the temperature and lighting of the algae, but instead rely on outside conditions, causing the growth rates of the algae to vary and making open pond type systems undesirable for some climates, including colder climates. Additionally, these operations can be vulnerable to contamination from other micro-organisms or other types of algae because of the openness to the environment. A further shortcoming of these open pond grow operations is that the depth of the ponds is typically kept relatively shallow to allow for sufficient penetration of the natural light.

Other approaches to growing algae include a clear, hanging bag and the clear plastic tube technologies. However, there are many innate problems associated with these approaches, primarily relating to durability and maintenance issues. Algae residue can build up inside the bags and block out sunlight, reducing the bags photometric grow efficiency; as a result the bags have to be regularly changed. The same fouling problems may occur in the clear plastic tubes. Additionally, the clear plastic tubes may degrade over time and have a limited life expectancy when exposed continuously to sunlight. This makes it necessary to replace the tubes every four or five years.

Additionally, many of these systems require an extensive and permanent installation to be constructed to provide the necessary infrastructure to support growth and harvesting operations, which can increase the land usage requirements.

Alternatives to the current state of the art for growing and harvesting algae are of interest as they may provide novel apparatus, methods and systems for growing and harvesting algae. In particular, there is a need for an apparatus for algae growth that is not vulnerable to weather extremes and changes, and that permits a large volume of algae production.

### SUMMARY OF INVENTION

In accordance with a broad aspect of the present invention, there is provided apparatus for growing algae, comprising: a tank for containing growth media, the tank having a substantially cylindrical body extending from a first end to a second end along a tank axis that is oriented horizontally, and an open top; a lid for closing the open top, the lid being at least partially transparent; and an impeller within the tank including: a barrel extending substantially horizontally between the first end and the second end, the barrel defining a barrel axis about which the impeller rotates, the barrel axis being positioned below and substantially parallel to the tank axis; and a plurality of vanes coupled to the barrel and extending substantially radially from the barrel.

In accordance with another broad aspect of the present invention, there is provided a method for growing algae, comprising swirling a growth media in a tank such that the growth media circulates around the tank passing through a dark section of the tank and an illuminated section of the tank.

It is to be understood that other aspects of the present invention will become readily apparent to those skilled in the art from the following detailed description, wherein various embodiments of the invention are shown and described by way of illustration. As will be realized, the invention is capable of other and different embodiments and its several details are capable of modification in various other respects, all within the present invention. Furthermore, the various embodiments described may be combined, mutatis mutandis, with other embodiments described herein. Accordingly, the drawings and detailed description are to be regarded as illustrative in nature and not as restrictive.

### BRIEF DESCRIPTION OF THE DRAWINGS

Referring to the drawings, several aspects of the present invention are illustrated by way of example, and not by way of limitation, in detail in the figures, wherein:

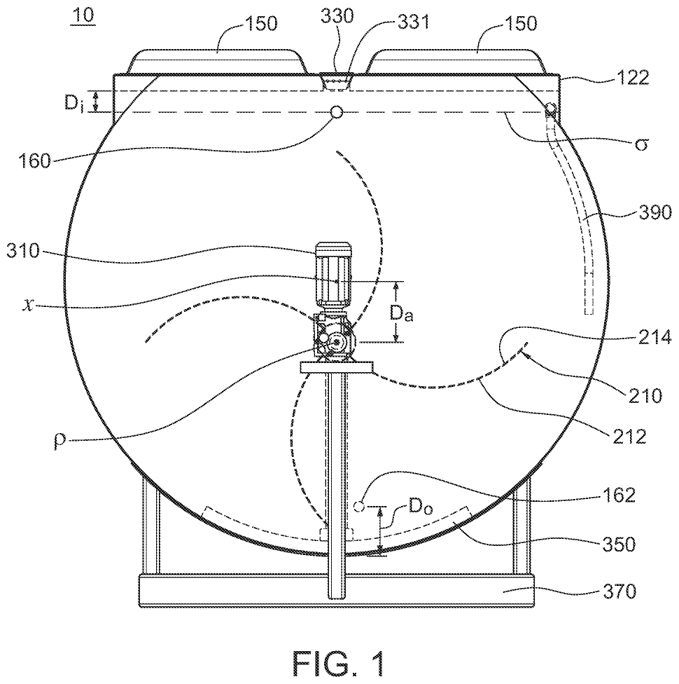

(a) FIG. 1 is a front elevation view of an algae growth apparatus according to one embodiment;

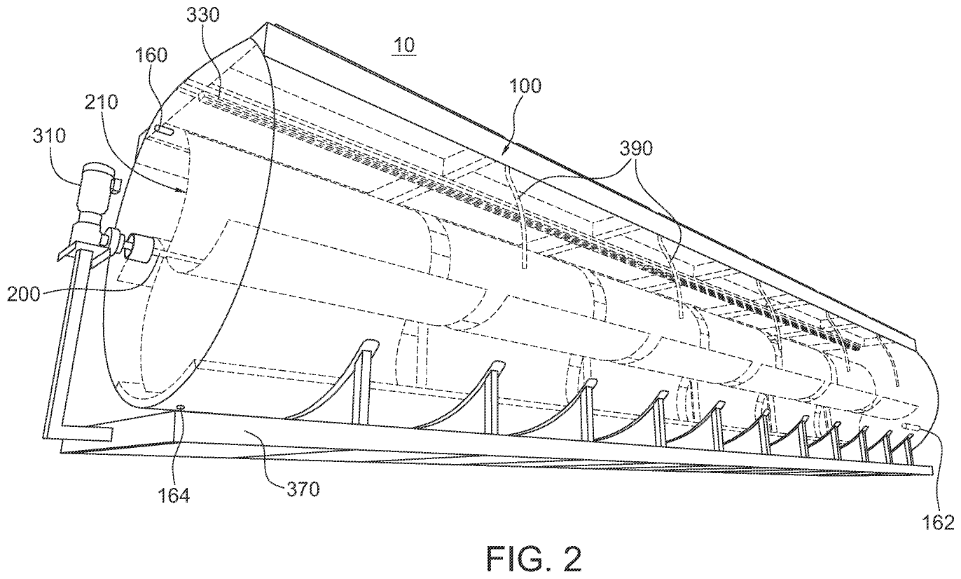

(b) FIG. 2 is a side perspective view of the algae growth apparatus of FIG. 1 with the side and end walls shown in phantom;

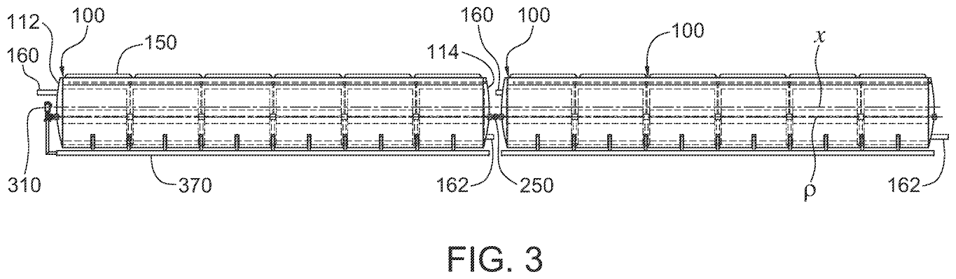

(c) FIG. 3 is a side elevation view of a first algae growth apparatus connected to a second algae growth apparatus according to another embodiment of the present invention. The side walls are shown in phantom to illustrate the impellers therewithin;

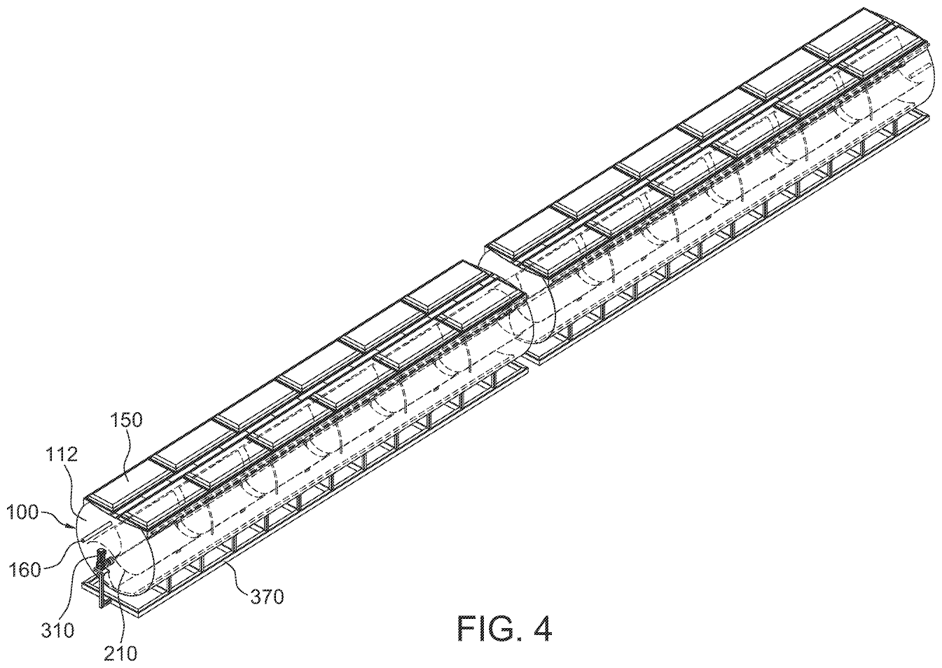

(d) FIG. 4 is a side perspective view of the first and second algae growth apparatus of FIG. 3. The side and end walls are shown in phantom to illustrate the impellers therewithin; and

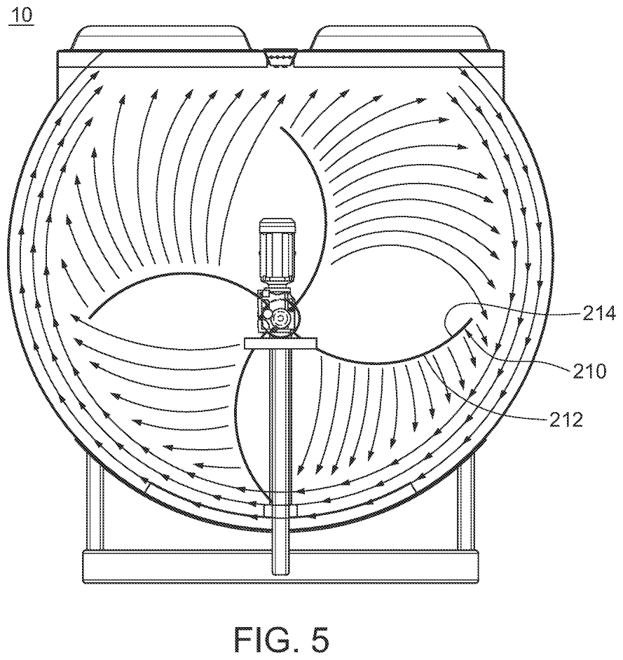

(e) FIG. 5 is a front elevation view of an algae growth apparatus according to the embodiment of FIG. 1, with arrows indicating fluid flow according to one of many possible operational embodiments.

### DETAILED DESCRIPTION OF EMBODIMENTS

The detailed description set forth below in connection with the appended drawings is intended as a description of various embodiments of the present invention and is not intended to represent the only embodiments contemplated by the inventor. The detailed description includes specific details for the purpose of providing a comprehensive understanding of the present invention. However, it will be apparent to those skilled in the art that the present invention may be practiced without these specific details.

In one embodiment, an apparatus for growing algae may include a substantially cylindrical tank for containing and growing algae. There may be a light transparent lid on the tank to expose the growing algae within the tank to light. The apparatus may include an agitation assembly with one or more impellers within the tank for circulating the contents of the tank. Each impeller may have one or more radially extending vanes disposed on a barrel hub within the tank. The barrel extends substantially horizontally parallel to long central axis of the tank. Multiple, for example four, impeller vanes may be disposed on the barrel.

With reference to FIGS. 1-4, and in particular FIG. 1, an apparatus 10 for growing algae may include a tank 100, a lid and an impeller with a barrel hub 200, and an impeller vane 210. Tank 100 may be substantially cylindrical and positioned horizontally with a long center axis χ that extends from a first end 112 to a second end 114. Each end may be closed by an end wall such that the tank can contain liquid material. The tank may have an inlet 160 for ingress of a growth media 400 into the tank (for example nutrients and seed algae), and an outlet 162, for egress of processed material from the tank (for example, grown algae and possibly residual matter from the growth media) for use and/or further processing. For example, inlet 160 may be positioned near the top of the tank, for example a first distance Di from the top of the tank; and outlet 162 may be positioned near the bottom of the tank, for example a second distance Do from the bottom of the tank. The first and second distances Di and Do may be selected to facilitate filling and emptying the tank to desired levels. For example, the first distance Di may be selected to allow filling up to the inlet's level indicated in FIG. 1 as a water line G, for example to 80% of its volume, and draining the tank down to the outlet's level, for example 20% of the tank's volume. The remaining material may act as seed material (i.e., inoculate material) for the next batch of algae to be grown. A drain 164, for example positioned at a floor of the tank, may be used to substantially completely drain the tank. The speed of the impeller of the agitation assembly (discussed further below) may be selected to allow the water line to remain substantially level. The inlet, outlet, and/or drain may be assisted by gravity and possibly additionally or alternatively by pumps.

Apparatus 10 may have an upper opening 120. The open top extends side to side, less than the diameter of the tank. A plane defined by the open top is substantially parallel to, but offset from, the long center axis χ.

A lid 150 is coupled to tank 100 to cover the open top. The lid may be transparent, thereby allowing light (for example, natural light) to reach the contents of the tank. In one embodiment, the lid is the only transparent enclosure part of the tank, such that external light is limited to entering the tank through the lid.

The lid may include one or more lid segments 150. As illustrated, the lid segments may be positioned on the top of the tank and may be arranged in two rows, along the length of the tank. For example, there may be a middle beam with a row of lid segments extending along each side of the middle beam. An upper portion of the tank may be configured to accommodate the shape of the lid. As illustrated, an open top 120 of the tank is flat and includes raised shoulders 122 extending up from the cylindrical side walls to accommodate the width of the lid segments. Alternatively, the shoulders could be removed by having the lid positioned closer to the tank axis χ, which would reduce the internal volume of the tank. In one embodiment, the lid segments may be opened and closed independently of one another. The distance from the interior surface of the lid to the water line G may define first distance Di. For example, Di may be substantially 2-6″ (5-15 cm), such as substantially 4″ (10 cm).

A light source 330 may be coupled to the tank to direct light into the contents of the tank to promote algae growth. This light source may be used in addition to, and/or instead of, natural light. In one embodiment, it is desired to ensure that there are lighted areas and dark areas within the tank. In particular, it is desired that light energy to the tank comes from the top. Therefore, the light source is positioned near the open top, where natural light would in any event enter the tank. The light source can be positioned adjacent the open top.

In the illustrated embodiment, light source 330 is an elongate LED panel positioned adjacent the open top at the upper end of the tank. In particular, the light source is installed between, and extending parallel with, the two rows of lid segments 150. This positions the light source centrally at the top of the tank. The light source may be operable as the only light source, in addition to natural light coming through the open top/transparent lids, within the tank. The light source is positioned within the volume of the tank to emit light down from the underside of the middle beam and above the water line. The light source 330 may be temperature controlled, for example by a water cooling device 331 coupled to the light source.

If there are other lights within tank, such as for observation, light source 330 near the open top is operable alone apart from the other lights so that illumination within the tank can be controlled to be only from the top.

Apparatus 10 may include an agitation assembly for agitating the contents of the tank. The agitation assembly may include one or more impellers installed on a drive shaft. Each impeller includes a barrel hub 200 driven by the drive shaft. The impellers are lined up along the tank such that their barrels are aligned and define a barrel axis ρ extending parallel to tank axis χ within the tank. The barrel hubs, and thereby the impellers, rotate about barrel axis ρ.

Barrel axis ρ may be offset from tank axis χ, for example, barrel axis ρ may be positioned a distance Da vertically lower than tank axis χ. In other words, the tank and the barrel may be non-concentric with the agitation assembly in the lower portion of the tank. The agitation assembly may be positioned such that vanes are closer to the bottom than to the top opening of the tank, such that the vanes sweep close to the bottom of the tank: closer to the bottom of the tank than to the top of the tank. Such an arrangement allows an upper layer of the contents of the tank to be exposed to light without the impeller blocking such light.

The barrel may be driven by a motor 310, which may be positioned on an exterior of the tank, for example at first end 112 of the tank. One or more impellers 210, each with one or more vanes, may be coupled to the barrel. As illustrated, the apparatus may include one to eight impellers. Where there is more than one impeller, they are positioned side by side along the barrel axis with a small space between the vane ends on adjacent impellers. Each impeller may be substantially continuous and consistent in cross section along its length. The impellers may be rotated together about the barrel axis, in substantial unison. While there may be more than one impeller in the tank between the end walls, the impellers may effectively act as a substantially single structure.

Each impeller in the illustrated embodiment, has four vanes 210, where each vane is positioned substantially equally apart from the next, i.e., substantially 90 degrees apart in an embodiment with four impellers. Each impeller vane may extend substantially radially from a base end at the barrel to an outboard, tip end spaced away from the barrel. Each impeller vane may be curved from its base end to its tip end, having a convex face 212 on one side and a concave (i.e. trough-like) face 214 on the other side. In one embodiment, the curvature is substantially consistent along the length of each vane. As such, each vane has a consistent cross sectional shape (orthogonal to axis x) along its full length.

All vanes on the impeller curve in the same direction. In operation, the impeller may be rotated about the barrel axis such that convex face 212 may be driven to be the leading face, and concave face 214 may thereby be the trailing face of the impeller vanes. This rotation is clockwise in FIG. 5. When so driven, the cylindrical shape of the tank and the rotating impellers cooperate to create centrifugal fluid dynamics that force the contents of the tank (i) to move radially away from the barrel axis and (ii) to circulate about the tank with the direction of the impeller, as illustrated by the arrows in FIG. 5. The contents of the tank are thereby swirled around the tank, and contents that may otherwise settle at the bottom of the tank are lifted and propelled to circulate around the wall of the tank, with contents near the lid being exposed to light. The algae in the contents of the tank may undergo photosynthesis near the open top, supported by the light energy from the light source and/or lid. However, over time even the contents at the waterline, closest to the lid, are gently circulated, mixed and drawn down with rotation of the impeller to be replaced by contents circulating up from the bottom of the tank.

In one embodiment, the tank may be made of a material that is non-transparent to light, such that the contents of the tank are only exposed to light from the lid and/or light source. In other words, during operation, the contents of the tank are centrifugally circulated around the tank, passing by an illuminated section of the tank (proximate the lid and/or light source), and passing by a dark section of the tank (that part of the tank that is non-transparent). The cylindrical side walls, cylindrical bottom and end walls of the tank may all be non-transparent to light.

The impeller including the vanes may also be constructed of material that is non-transparent to light, such that the contents of the tank below the impeller is shaded from exposure to light passing through the lid and/or from the light source. Because the impeller is non-concentric, closer to the bottom of the cylindrical tank, there remains an upper layer of the contents of the tank that is above the impeller and thereby exposed to light. The space above the impeller defines a volume greater than the volume of the tank below the impeller, so overall more volume of the tank receives light than that volume not receiving light input.

This configuration, together with the speed of rotation of the impellers, may allow the apparatus to control the amount of light to which algae is exposed. This may be advantageous because excess light exposure may cause algae to deteriorate. That is, organisms of the algae may become bleached by too much light. It will be appreciated that such an embodiment is advantageous over prior art systems that grow algae in a lake or pond, which expose only the top few inches of material to light for an uncontrolled period of time.

As mentioned above, the speed of the impeller of the agitation assembly may be selected to allow the water line to remain substantially level. For example, the barrel may rotate at a rate of 15 revolutions per minute (rpm) or less, for example 10 rpm or 5 rpm. The size of the lid, the speed of rotation, and the power of the light source, among other factors, or any one or more of such factors, may be selected to control the amount of light to which the contents of the tank are exposed. In one embodiment, a given segment of the tank's circulating contents is exposed to light for substantially 0.5-2 seconds (for example 0.4 to 0.8 or about 0.6 of a second) followed by being shielded from light for substantially 0.5-3 seconds (for example 0.8 to 1.2 seconds or about 1 second).

An additive line, herein called an atomizer 390, may be coupled to the tank for emitting fluid, such as gas, through an outlet end into the tank. Such fluids may include any one or more of carbon dioxide, oxygen, water, phosphorous, nitrogen, and/or other nutrients selected to promote algae growth. For example, atomizer 390 may include a conduit that enters the tank, for example through the open top, and extends along an interior wall of the tank, extending substantially along a side wall of the tank perpendicular to the tank axis. The conduit terminates in an outlet end that is positioned below the water line. There may be multiple atomizers spaced apart along the length of the tank. The atomizer may be configured to deliver nutrients to the tank to promote algae growth.

From the description above and FIG. 5, it will be appreciated that the impeller has a downwardly sweeping side and an upwardly sweeping side. The downwardly sweeping side is on the right hand side in FIG. 5, where the vanes are driven down and act to push the contents of the tank down away from the upper opening and under the impeller. In one embodiment, at least one atomizer 390 is positioned within the tank with its outlet end on the side of the tank where the impeller sweeps downwardly. As such, the introduced fluids are swept down by the impeller with the tank contents across the bottom of the tank, under the impeller before being circulated up towards the waterline. Thereby, the residence time of introduced fluids is maximized since the introduced fluids are forced to remain entrained in the tank contents at least until the contents are swept up toward the opening.

The atomizers may be located to line up with the small spaces between adjacent impellers within the tank.

A heat source 350, for example a heat exchanger, may be positioned on the bottom of the interior of the tank. The heat source may be used to control the temperature of the contents of the tank to promote algae growth. The heat source may alternatively be positioned elsewhere, for example at a side of the tank.

A support structure, for example a skid 370, may be coupled to the tank to hold it in place during operation.

In one embodiment, multiple tanks may be connected to each other. It is to be appreciated that the various structures described in this specification may be modular, portable, and/or linkable. As shown in FIGS. 3 and 4, two tanks 100 are connected to each other by connection 250. Connection 250 may connect the barrel of the first tank to the barrel of the second tank, such that one motor 310 can drive both barrels. Accordingly, the tanks may be aligned end-to-end and may thereby be coaxial with one another. This modular aspect may be beneficial for shipping, maintenance, and scaling purposes.

### CLAUSES

Clause 1. An apparatus for growing algae, comprising: a tank for containing growth media, the tank having a substantially cylindrical body extending from a first end to a second end along a tank axis, and an open top; a lid for closing the open top, the lid being at least partially transparent; a barrel, coupled to the tank, the barrel extending from the first end to the second end, the barrel having a barrel axis below and substantially parallel to the tank axis; and an impeller coupled to the barrel and extending substantially radially from the barrel.

Clause 2. The apparatus of any one or more of clauses 1-7, wherein the impeller is curved.

Clause 3. The apparatus of any one or more of clauses 1-7, further comprising a second impeller, a third impeller, and a fourth impeller.

Clause 4. The apparatus of any one or more of clauses 1-7, further comprising a light source.

Clause 5. The apparatus of any one or more of clauses 1-7, further comprising a heat source.

Clause 6. The apparatus of any one or more of clauses 1-7, further comprising an atomizer.

Clause 7. A method for growing algae, comprising swirling a growth media in a tank such that the growth media circulates around the tank passing by a dark section of the tank and an illuminated section of the tank.

The previous description of the disclosed embodiments is provided to enable any person skilled in the art to make or use the present invention. Various modifications to those embodiments will be readily apparent to those skilled in the art, and the generic principles defined herein may be applied to other embodiments without departing from the spirit or scope of the invention. Thus, the present invention is not intended to be limited to the embodiments shown herein, but is to be accorded the full scope consistent with the claims, wherein reference to an element in the singular, such as by use of the article “a” or “an” is not intended to mean “one and only one” unless specifically so stated, but rather “one or more”. All structural and functional equivalents to the elements of the various embodiments described throughout the disclosure that are known or later come to be known to those of ordinary skill in the art are intended to be encompassed by the elements of the claims. Moreover, nothing disclosed herein is intended to be dedicated to the public regardless of whether such disclosure is explicitly recited in the claims. No claim element is to be construed under the provisions of 35 USC 112, sixth paragraph, unless the element is expressly recited using the phrase “means for” or “step for”.

1. An apparatus for growing algae, comprising:
a tank for containing growth media, the tank having
a substantially cylindrical body extending from a first end to a second end along a tank axis that is oriented horizontally, and
an open top;
a lid for closing the open top, the lid being at least partially transparent; and
an impeller within the tank including:
a barrel extending substantially horizontally between the first end and the second end, the barrel defining a barrel axis about which the impeller rotates, the barrel axis being positioned below and substantially parallel to the tank axis; and
a plurality of vanes coupled to the barrel and extending substantially radially from the barrel.

2. The apparatus of claim 1, wherein each of the plurality of vanes has a base end where the vane is attached to the barrel and a tip end opposite the base end and each of the plurality of vanes is curved from the base end to the tip end to define a convex face and a concave face.

3. The apparatus of claim 2, wherein the impeller is configured to rotate with the convex faces of the plurality of vanes leading.

4. The apparatus of claim 1, further comprising a light source adjacent the open top.

5. The apparatus of claim 1, wherein the tank and the impeller are constructed of material non-transparent to light, to thereby create a dark area in the tank below the impeller.

6. The apparatus of claim 1, further comprising a heat source configured to apply heat energy to an interior volume of the tank.

7. The apparatus of claim 1, further comprising an atomizer having an outlet end within the tank.

8. The apparatus of claim 7, wherein the impeller is configured to rotate downwardly on one side away from the open top and the atomizer is positioned with the outlet end adjacent the downwardly rotating side.

9. A method for growing algae in an apparatus for growing algae according to claim 1, comprising swirling a growth media in the tank such that the growth media is circulated down away from the open top, through a dark section of the tank below the impeller and then into an illuminated section of the tank nearer the open top.

10. The method of growing algae of claim 9 wherein the growth media is illuminated only through the open top or a light source mounted adjacent the open top.

11. The method of growing algae of claim 9 further comprising injecting a fluid into the growth media only when the growth media is circulated down away from the open top.
